(19) United States
(12) Patent Application Publication
(10) Pub. No.: US 2023/0000379 A1
(43) Pub. Date: Jan. 5, 2023

(54) GARMENT CUFF FOR DETECTING PHYSIOLOGICAL DATA

(71) Applicant: MYANT INC., Toronto (CA)

(72) Inventors: Tony CHAHINE, Toronto (CA); Parth Satvik JAIN, Toronto (CA); Calvin Fook-Lam KWOK, Toronto (CA); Dan Catalin CRISTEA, Toronto (CA); Senafa NOUDEHOU, Toronto (CA)

(21) Appl. No.: 17/790,924

(22) PCT Filed: Dec. 30, 2020
(86) PCT No.: PCT/CA2020/051808

§ 371 Date: Jul. 5, 2022

Related U.S. Application Data
(60) Provisional application No. 62/957,606, filed on Jan. 6, 2020.

(51) Int. Cl. A61B 5/024; A61B 5/00; A61B 5/0245; A61B 5/0295; A61B 5/11; A61B 5/026

(52) U.S. Cl. CPC A61B 5/02438; A61B 5/6804; A61B 5/0245; A61B 5/0295; A61B 5/1102; A61B 5/0261

(57) ABSTRACT

A garment cuff for detecting physiological data. The garment cuff includes: an elongate band having a first region and a second region and defining an adjustable loop for receiving a user limb based on the first region being positioned proximal the second region. The garment cuff includes a stiffener member coupled to the first region to define a substantially rigid base and a sensor panel affixed to the substantially rigid base on a user limb facing side of the first region. The garment cuff includes a fastener coupled to the elongate band and configured to position the sensor panel against the user limb with substantially consistent pressure.

### CROSS REFERENCE TO RELATED APPLICATIONS

This application claims priority from U.S. provisional patent application No. 62/957,606, entitled “GARMENT CUFF FOR DETECTING PHYSIOLOGICAL DATA”, filed on Jan. 6, 2020, the entire contents of which are hereby incorporated by reference herein.

### FIELD

Embodiments of the present disclosure generally relate to the field of smart garments, and in particular to a garment cuff for detecting physiological data.

### BACKGROUND

Specialized apparatus or devices for measuring physiological data, such as blood pressure, may be secured to a patient user during physiological data acquisition. For example, a sphygmomanometer in combination with a stethoscope may be configured to determine blood pressure of a patient user. The sphygmomanometer may include an inflatable cuff to collapse and subsequently release a patient user's artery in a controlled manner for determining blood pressure of the patient user. Such specialized equipment may not be intended for continuous wear.

### SUMMARY

In one aspect, the present disclosure provides a garment cuff for detecting physiological data. The garment cuff may include: an elongate band having a first region and a second region and defining an adjustable loop for receiving a user limb based on the first region being positioned proximal the second region; a stiffener member coupled to the first region to define a substantially rigid base; a sensor panel affixed to the substantially rigid base on a user limb facing side of the first region; and a fastener coupled to the elongate band and configured to position the sensor panel against the user limb with substantially consistent pressure.

In some embodiments, the stiffener member may be knitted to the first region of the elongate band.

In some embodiments, the fastener may include: an elastomeric band wrapping around the elongate band and a latch coupled to the elastomeric band. The latch may transition the elongate band between a first adjustable loop circumference and a second adjustable loop circumference.

In some embodiments, the sensor panel may include a combination of data acquisition sensors including an electrocardiogram (ECG) sensor and a photoplethysmogram (PPG) sensor.

In some embodiments, the sensor panel may include a ballistocardiography (BCG) sensor.

In some embodiments, the garment may include a two-way stretch fabric coupled to the second region.

In some embodiments, the garment may include a 1-way stretch fabric joining the first region and the second region, wherein the 1-way stretch fabric stretches in a direction between the first region and the second region.

In some embodiments, the garment may include a data acquisition module coupled to the sensor panel for receiving physiological data from the sensor panel.

In some embodiments, the data acquisition module may be removably mounted on a bridge region between the first region and the second region of the elongate band.

In some embodiments, the garment may include a shirt, and the cuff may be coupled to a shirt sleeve of the shirt.

In some embodiments, the garment may include at least one of pants or shorts, and the cuff may be coupled to a pant leg.

In another aspect, the present disclosure provides a garment for detecting physiological data. The garment includes a garment body; and a cuff coupled to the garment body providing a garment opening for receiving a user limb. The cuff may include the garment cuff described in the present disclosure.

In another aspect, a non-transitory computer-readable medium or media having stored thereon machine interpretable instructions which, when executed by a processor may cause the processor to perform one or more methods described herein.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrates a system for detecting physiological data, in accordance with an embodiment of the present disclosure;

FIG. 2 illustrates a plan view of a shirt yoke, in accordance with an embodiment of the present disclosure;

FIG. 3 illustrates an enlarged, partial view of the first sleeve portion of FIG. 2;

FIGS. 4A and 4B illustrate enlarged, elevation views of the first sleeve portion of FIG. 2;

FIG. 5 illustrates an enlarged, elevation view of the first sleeve portion of FIG. 2;

FIGS. 6A and 6B illustrate a perspective view and a top plan view, respectively, of a latch for a garment, in accordance with an embodiment of the present disclosure;

FIG. 7A illustrates a side elevation view of the latch of FIG. 6A;

FIG. 7B illustrates a cross-sectional view of the latch taken at line A-A of FIG. 6B;

FIGS. 8A and 8B illustrate side elevation views of the latch of FIG. 6A;

FIG. 9 illustrates the latch of FIG. 8B being transitioned from a closed state to an open state;

FIGS. 10A and 10B illustrate perspective views of a user conducting operations for calibrating a garment for detecting physiological data, in accordance with embodiments of the present disclosure;

FIG. 11A illustrates a user wearing a garment for detecting physiological data, in accordance with an embodiment of the present disclosure;

FIG. 11B illustrates a partial view of the user transitioning the latch from an open state to a closed state;

FIG. 12A illustrates a cross-sectional end view of an assembled first sleeve portion of FIG. 2, in accordance with an embodiment of the present disclosure;

FIG. 12B illustrates a partial, perspective view of an assembled first sleeve portion of FIG. 2, in accordance with an embodiment of the present disclosure; and

FIG. 13 illustrates a block diagram of a computing device, in accordance with an embodiment of the present disclosure.

### DETAILED DESCRIPTION

Specialized devices may be configured for determining physiological statistics of a user. For example, a combination of a sphygmomanometer and a stethoscope may be used for determining a user's blood pressure. The sphygmomanometer may include an inflatable cuff for collapsing a user's artery and, subsequently, releasing the user's artery in a controlled manner for determining blood pressure of the patient user. Upon collapsing and releasing the patient user's artery, the stethoscope may be used to determine at what pressure blood begins flowing in the artery, and at what pressure the blood flow becomes unimpeded. Such specialized equipment and methods for measuring blood pressure may not be worn for extended periods of time and, thus, may not be suitable for obtaining numerous blood pressure measurement readings over time. Further, such specialized equipment may be invasive or uncomfortable to the user. The user may experience discomfort as the inflatable cuff may be used to collapse an artery, preventing blood flow. Less invasive devices for acquiring physiological statistics over a duration of time may be desirable.

In some embodiments of the present disclosure, devices or apparatus for determining physiological statistics, such as blood pressure, may be provided in a garment. The garment may be a t-shirt or a long sleeve shirt having one or more sleeves for receiving a patient user's arms. At least one shirt sleeve may include a sensor array configured to be secured, via consistent pressure, to the patient user's arm. In some embodiments, the sensor array may be secured to the user's arm in a consistent or repeatable way, such that the device for determining physiological statistics may collect a plurality of blood pressure readings over time. Because example devices may collect blood pressure readings over time, in some scenarios, trends and deviations therefrom may be determined.

Examples described in the present disclosure may be directed to measuring blood pressure based on physiological data acquisitions using a sensor array secured to a user arm. It may be appreciated that devices for measuring other physiological statistics based on one or more sensor arrays secured, via consistent pressure, to any other type of user limb or body part may be contemplated. Embodiments described in the present disclosure may be directed to shirts and shirt sleeves. It may be appreciated that the apparatus and devices for acquiring physiological data may be provided for other types of garments, such as pants, hats, or other types of garments that may receive a user limb or a part of the user's body.

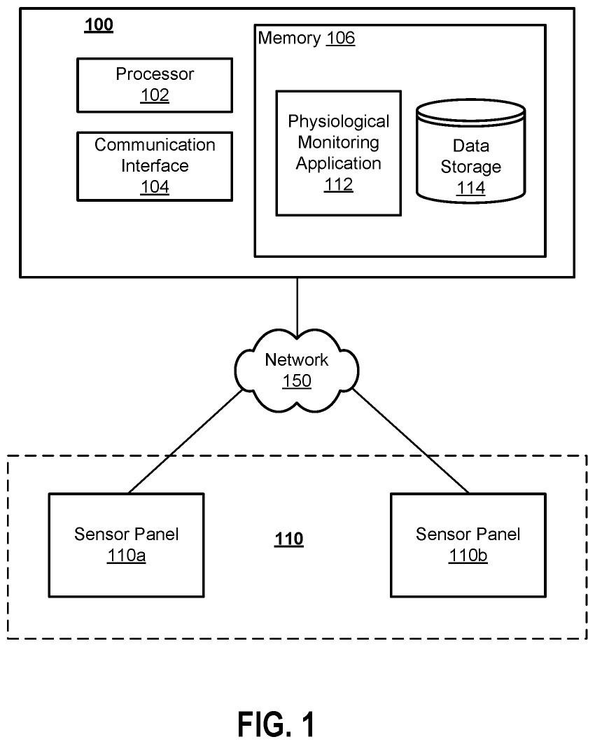

Reference is made to FIG. 1, which illustrates a system for detecting physiological data, in accordance with an embodiment of the present disclosure. The system may include a controller device 100 and one or more sensor panels 110. In some embodiments, the one or more sensor panels 110 may be affixed to a garment, and the one or more sensor panels 110 may be positioned against a user's skin for detecting physiological data. In some embodiments, the controller device 100 may be a computing device for transmitting or receiving data messages to or from the one or more sensor panels 110.

The controller device 100 may be coupled to the at least one sensor panels 110 via a network 150. The network 150 may include any wired or wireless communication path, such as an electrical circuit. In some embodiments, the network 150 may include one or more busses, interconnects, wires, circuits, and/or any other connection and/or control circuit, or a combination thereof. In some embodiments, the network 150 may include a wired or a wireless wide area network (WAN), local area network (LAN), a combination thereof, or the like. In some embodiments, the network 150 may include a Bluetooth® network, a Bluetooth® low energy network, a short-range communication network, or the like. The network 150 may be a communication interface such that the controller device 100 and the at least one sensor panel 110 may communicate.

In some embodiments, the system illustrated in FIG. 1 may be integrated into a garment, such as a t-shirt, a long sleeve shirt, or a garment that may be worn by a user. For example, a t-shirt may be an athletic shirt. In the example of FIG. 1, the sensor panels 110 may include a first sensor panel 110a and a second sensor panel 110b. The first sensor panel 110a may be affixed to a portion of a first shirt sleeve on a user facing side such that, when a user wears the garment, the first sensor panel 110a may be configured to contact the user's arm. The second sensor panel 110b may be affixed to a portion of a second shirt sleeve on a user facing side such that, when a user wears the garment, the second sensor panel 110b may be configured to contact the user's arm. Although two sensor panels 110 are illustrated in FIG. 1, any number of sensor panels 110 may be contemplated. In some embodiments, one or more of the sensor panels 110 may be affixed to a cuff of a shirt sleeve, and when a user limb is received within the cuff of the shirt sleeve, one or more of the sensor panels 110 may be positioned for contacting the user's arm.

In some embodiments, the controller device 100 may be integrated into the garment and may be coupled to the sensor panels 110 via electrical interconnection means, such as an electrical circuit. In some embodiments, the controller device 100 may be removably mounted to the garment, such that the controller device 110 may be removed when the garment is cleaned or laundered. The controller device 100 may receive one or more physiological data sets from the one or more sensor panels 110 and may conduct operations for analyzing the one or more physiological data sets for determining physiological statistics, such as blood pressure.

In some embodiments, the garment may be a smart garment formed of a knitted textile. In some embodiments, the garment may be formed of other textile forms and/or techniques such as weaving, knitting (warp, weft, etc.) or the like. In some embodiments, the smart garment may include one of a knitted textile, a woven textile, a cut and sewn textile, a knitted fabric, a non-knitted fabric, in any combination and/or permutation thereof. Example structures and interlacing techniques of textiles formed by knitting and weaving are disclosed in U.S. patent application Ser. No. 15/267,818, the entire contents of which are herein incorporated by reference.

As used herein, “textile” refers to any material made or formed by manipulating natural or artificial fibres to interlace to create an organized network of fibres. Generally, textiles are formed using yarn, where yarn refers to a long continuous length of a plurality of fibres that have been interlocked (i.e., fitting into each other, as if twined together, or twisted together). Herein, the terms fibre and yarn are used interchangeably. Fibres or yarns can be manipulated to form a textile according to any method that provides an interlaced organized network of fibres, including but not limited to weaving, knitting, sew and cut, crocheting, knotting and felting.

Different sections of a textile can be integrally formed into a layer to utilize different structural properties of different types of fibres. For example, conductive fibres can be manipulated to form networks of conductive fibres and non-conductive fibres can be manipulated to form networks of non-conductive fibers. These networks of fibres can comprise different sections of a textile by integrating the networks of fibres into a layer of the textile. The networks of conductive fibres can form one or more conductive pathways that electrically connect sensors and actuators embedded in the smart garment, for conveying data and/or power to and/or from these components.

In some embodiments described in the present disclosure, the sensors embedded in the smart garment may be the one or more sensor panels 110 for detecting physiological data. The network 150 may include the network of conductive fibres of the smart textile for conveying data and/or power between the one or more sensor panels 110 and the controller device 100.

In some embodiments, multiple layers of textile may be stacked upon each other to provide a multi-layer textile.

In the present disclosure, “interlace” refers to fibres (either artificial or natural) crossing over and/or under one another in an organized fashion, typically alternately over and under one another, in a layer. When interlaced, adjacent fibres touch each other at intersection points (e.g., points where one fibre crosses over or under another fibre). In one example, first fibres extending in a first direction can be interlaced with second fibres extending laterally or transverse to the fibres extending in the first connection. In another example, the second fibres can extend laterally at 90° from the first fibres when interlaced with the first fibres. Interlaced fibres extending in a sheet can be referred to as a network of fibres.

In the present disclosure, “integrated” or “integrally” refers to combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more seconds comprising networks of fibres are said to be “integrated” together into a textile (or “integrally formed”) when at least one fibre of one network is interlaced with at least one fibre of the other network such that the two networks form a layer of the textile. Further, when integrated, two sections of a textile can also be described as being substantially inseparable from the textile. Here, “substantially inseparable” refers to the notion that separation of the sections of the textile from each other results in disassembly or destruction of the textile itself.

In some examples, conductive fabric (e.g., group of conductive fibres can be knit along with (e.g., to be integral with) the base fabric (e.g., surface) in a layer. Such knitting may be performed using a circular knit machine or a flat bed knit machine, or the like, from a vendor such as Santoni or Stoll.

The controller device 100 includes a processor 102 configured to implement processor readable instructions that, when executed, configure the processor 102 to conduct operations described herein. The controller device 100 includes a communication device 104 to communicate with other computing or sensor devices, to access or connect to network resources, or to perform other computing applications by connecting to a network (or multiple networks) capable of carrying data. In some examples, the communication device 104 may include one or more busses, interconnects, wires, circuits, and/or any other connection and/or control circuit, or combination thereof. The communication device 104 may provide an interface for communicating data between the controller device 100 and the one or more sensor panels 110. In some embodiments, the one or more busses, interconnects, wires, circuits, or the like may be the network of conductive and non-conductive fibers of a smart textile.

The controller device 100 may include memory 106. The memory 106 may include one or a combination of computer memory, such as static random-access memory (SRAM), random-access memory (RAM), read-only memory (ROM), electro-optical memory, magneto-optical memory, erasable programmable read-only memory (EPROM), and electrically-erasable programmable read-only memory (EEPROM), Ferroelectric RAM (FRAM) or the like.

The memory 106 may store a physiological monitoring application 112 including processor readable instructions for conducting operations described herein. In some examples, the physiological monitoring application 112 may include operations for receiving and storing physiological data of a user, such as blood pressure data, and may include operations to determine one or more blood pressure trends over time. By integrating the one or more sensor panels into a garment, embodiments of the present disclosure may be configured for a user to wear the garment for extended periods of time and for collecting physiological data with reduced discomfort from the positioning of the sensor panels against the user's limb.

The system 100 may include a data storage 114. In some embodiments, the data storage 114 may be a secure data store. In some embodiments, the data storage 114 may store received physiological data sets, such as blood pressure data, heart rate data, or other types of data. In some examples, the data storage 114 may store data associated with criteria for analyzing received physiological data sets. In some embodiments, the stored criteria may include blood pressure criteria that may be used for generating indications that blood pressure data may be trending beyond a defined blood pressure range.

In some embodiments, the sensor panels 110 may include one or more sensors, and the one or more sensors may include one or a combination of electrocardiogram (ECG) sensors, photoplethysmogram (PPG) sensors, ballistocardiography (BCG) sensors, or other types of sensors.

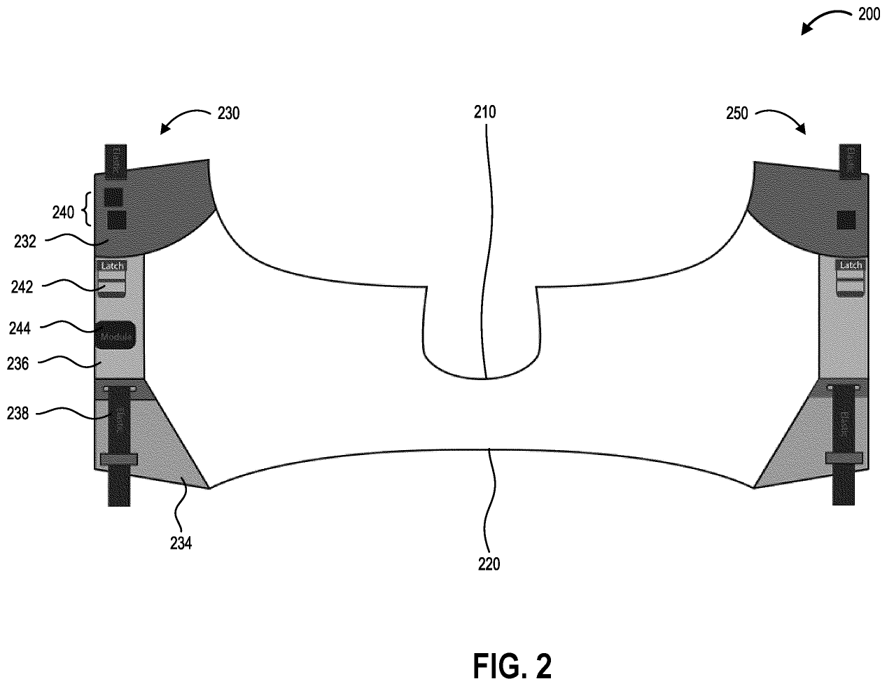

In examples described in the present disclosure, the sensor panels 110 may be integrated into a garment. In some scenarios, a garment having a given size may be suitable for users having a spectrum of arm circumferences. For example, a medium size garment t-shirt may be suitable to be worn by one user having an arm circumference of 25 centimeters, and may also be suitable to be worn by another user having an arm circumference of 40 centimeters. To obtain physiological sensor data readings in a repeatable way, it may be desirable to provide the garment for detecting physiological data having features to position a sensor panel against a user limb with substantially consistent pressure. In some scenarios, from the point of view of a user of embodiments described in the present disclosure, the sensor panels may be perceived to be pressed against the user limb without any temporary tightening during data acquisition (e.g., without any tightening of the garment that is akin to a sphygmomanometer inflating to collapse a user's artery during blood pressure measurements). To illustrate embodiments of the present disclosure, reference is made to FIG. 2.

FIG. 2 illustrates a plan view of a shirt yoke 200, in accordance with an embodiment of the present disclosure. The shirt yoke 200 may be a component of a garment body. The garment body may include a shaped pattern piece for forming a part of the garment that fits around a user's neck and shoulders. The shirt yoke 200 may include a neckline seam 210 and a back yoke seam 220.

In some embodiments, the shirt yoke 200 may include a first sleeve portion 230 and a second sleeve portion 250. In the example illustrated in FIG. 2, the first sleeve portion 230 may be assembled as a left sleeve of the t-shirt garment. The second sleeve portion 250 may be assembled as a right sleeve of the t-shirt garment. The first sleeve portion 230 or the second sleeve portion 250 may include features to configure the respective sleeves in one or more states, and may include features to position a sensor panel 240 against a user limb with substantially consistent pressure. In some embodiments, the sensor panel 240 may include features of or may be substantially similar to the one or more sensor panels 110 illustrated in FIG. 1.

In some embodiments, the first sleeve portion 230 may include a cuff coupled to the garment body, and the cuff may be configured for receiving a user limb. The cuff may include an elongate band having a first region 232 and a second region 234. As will be illustrated in the present disclosure, the elongate band may define an adjustable loop for receiving the user limb based on the first region 232 being positioned proximal to the second region 234. When the first sleeve portion 230 is configured or assembled as a sleeve, the first sleeve portion 230 may receive an arm of a user.

The first region 232 may include a stiffener member. The stiffener member may be coupled to the first region 232 to define a substantially rigid base. In some embodiments, the stiffener member may include semi-crystalline polyamide materials having high strength or high stiffness. In some examples, the stiffener member may include material known as Grilon®. The stiffener member material may be knitted into the first region 230. Other techniques or methods of integrating the stiffener member material into the first region 230, such as gluing, fusing, or the like, may be contemplated.

The first sleeve portion 230 may include the sensor panel 240 affixed to the substantially rigid base on a user limb facing side of the first region 232. When the first sleeve portion 230 is assembled to provide a sleeve for receiving a user limb, the sensor panel 240 may be configured to contact the user limb for detecting physiological data. In some embodiments, the sensor panel 240 may include one or a combination of sensors. The sensor panel 240 may include a electrocardiogram (ECG) sensor, a photoplethysmogram (PPG) sensor, or a ballistocardiography (BCG) sensor. Other types of sensors may be contemplated.

In some embodiments, the one or more sensors may generate physiological sensor data for determining physiological statistics associated with the user. In some examples, the physiological statistics may include blood pressure data. In some embodiments, the controller device 100 (FIG. 1) may conduct processor-executable operations to retrieve physiological data and to determine blood pressure data based on a combination of physiological data retrieved from the sensor panel 240.

As accuracy of sensed data or sensing ability of the sensor panel 240 may diminish when the sensor panel 240 experiences motion, the substantially rigid base provides a support base that may reduce stretching when the garment is worn by a patient user and when the sensor panel 240 is configured to contact the user's limb.

As described, the garment having a given size (e.g., size medium) may be worn by users having different arm circumferences. To configure the garment to reliably detect and generate physiological sensor data for users having different arm circumferences, the first sleeve portion may include fastener features for adapting the sleeve opening to the user limb. For example, the first sleeve portion 230 may include a fastener configuring the adjustable loop in at least two elongate band states. The first sleeve portion 230 may be configured in a closed state for securing the sensor panel 240 against the user limb via consistent pressure.

In some embodiments, the fastener may include an elastomeric band 238 configured to wrap around the elongate band. In some embodiments, the elastomeric band 238 may include gradations or size markings thereon for adapting the fastener to users having different arm circumference measurements. In some embodiments, when the first region 232 having the sensor panel 240 affixed to the substantially rigid base is partially overlapped by the second region 234, the elastomeric band may be tightened over the overlapping first region 230 and second region 234. The size markings of the elastomeric band may correspond to a series of arm circumference measurements. The garment user may tighten the elastomeric band over the overlapping first region 230 and second region 234 to configure the sensor panel 240 to contact the user arm with a determined and/or consistent pressure.

When the user desires to re-adjust or remove the garment (e.g., for laundering or cleaning), the first sleeve portion 230 may be configured to an open state to allow the user to remove or distance the user limb from the first sleeve portion 230. When the fastener is configured in an open state, the circumferential dimension of the adjustable loop defined by the first sleeve portion 230 may be increased, thereby allowing the user to remove the garment or to reposition the garment.

In some embodiments, the fastener may include a latch device 242 coupled to the elastomeric band 238. The latch device 242 may be configured to transition the first sleeve portion 230 from an open state to a closed state, or vice versa, in a consistent way. Continuing with the above-described example, the latch device 242 may be coupled based on a particular size marking provided on the elastomeric band 238, where the particular size marking may be a function of or correspond to the arm circumference measurement of the user. Thus, when the latch device 242 is positioned in the closed state, the first sleeve portion 230 may be configured to position the sensor panel 240 to contact the user limb with substantially consistent pressure.

When the latch device 242 is positioned in the open state, the adjustable loop defined by the first sleeve portion 230 may expand by an amount determined by the latch device 242, such that the sleeve may be loosened to allow the user to re-position the garment or to remove the garment. When the latch device 242 is positioned in the open state, the sensor panel 240 may be pulled away from the user limb.

In some embodiments, second region 234 may include or be constructed with a multi-way stretch fabric. In some embodiments, the second region 234 of the elongate band may include a mesh-like fabric and constructed of a two-way stretch fabric.

In some embodiments, the first sleeve portion 230 may include a bridge region 236 for interconnecting the first region 232 and the second region 234. In some embodiments, the bridge region 236 may be constructed of a rib-knit hem material, and may be constructed of a one-way stretch material that stretches in a direction substantially parallel to a direction from the first region 232 to the second region 234. Other material construction and stretch configurations of the bridge region 236 may be contemplated.

In the example illustrated in FIG. 2, the first sleeve portion may be configured to include a removably mounted computing module 244. The computing module 244 may be similar to the computing device 100 of FIG. 1, and may be configured to receive one or more physiological data sets from the sensor panel 240. In some embodiments, the computing module 244 may be configured to conduct operations for analyzing the physiological data sets for determining physiological statistics, such as blood pressure. In some embodiments, the computing module 244 may be removably coupled to the bridge region 236. The computing module 244 may be removed when the garment is cleaned or laundered.

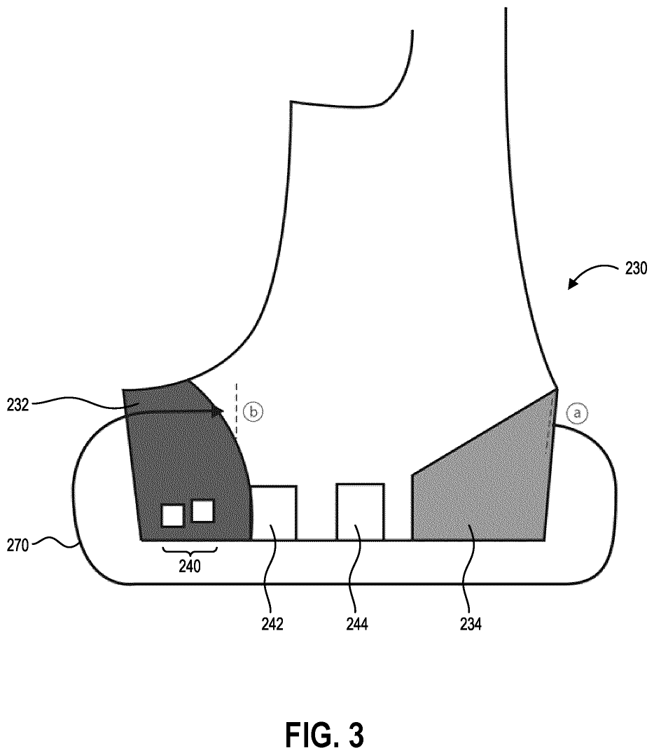

Reference is made to FIG. 3, which illustrates an enlarged, partial view of the first sleeve portion 230 illustrated in FIG. 2. In FIG. 3, the second region 234 may be configured to wrap around in a direction indicated by an arrow labelled with reference numeral 270. The second region 234 may be positioned proximal to or may over overlap the first region 232 for providing a garment sleeve from the first sleeve portion 230. The first region 232 may include a stiffener member coupled thereon to define a substantially rigid base, and the substantially rigid base may provide a support base for the sensor panel 240. As the accuracy or sensing ability of the sensor panel may diminish when the sensor panel 240 experiences motion, the substantially rigid base may provide a support base to reduce stretching or movement in the sensor panel 240 when the garment is worn by the user and/or when the sensor panel is in contact with the limb.

In some embodiments, when the second region 234 is wrapped around in the direction indicated by the arrow 270, an edge indicated by the letter (a) may be positioned proximal to the position indicated by the letter (b). The positioning of the edge of the second region 234 relative to the position indicated by the letter (b) may be a function of the circumferential arm measurement of the user and a required positioning via consistent pressure of the sensor panel 240 on the user arm. In some embodiments, the edge indicated by the letter (a) may be an underarm seam top-stitched down approximately one inch vertically along the position indicated by the letter (b).

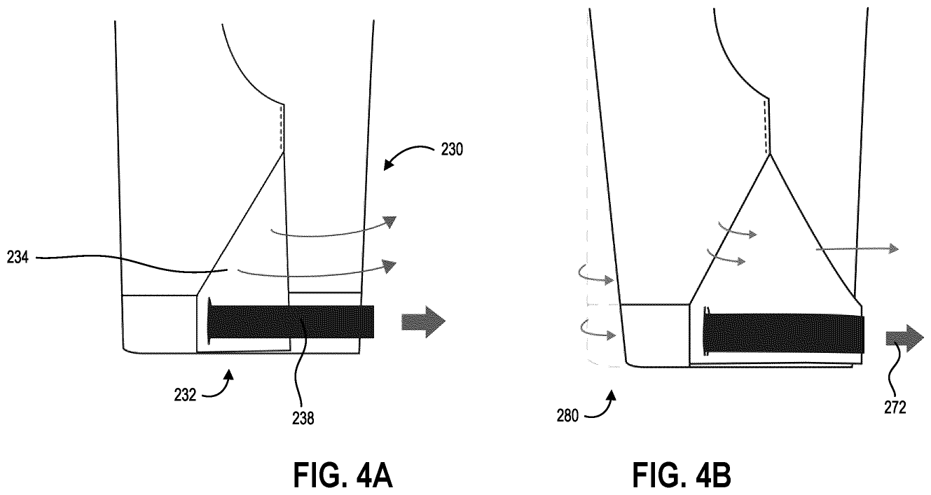

Reference is made to FIGS. 4A and 4B, which illustrate enlarged, elevation views of the first sleeve portion 230 of FIG. 2 assembled as a sleeve, in accordance with embodiments of the present disclosure. In FIG. 4A, the second region 234 may be positioned proximal to the first region 232 (e.g., in an overlapping formation) to define the adjustable loop for receiving the user limb.

In FIG. 4B, once the second region 234 is wrapped around to overlap at least a portion of the first region 232, the elastomeric band 238 (e.g., of a fastener) may be configured to reduce the circumferential dimension of the sleeve. When the fastener is adjusted in the direction of the arrow 272, the circumferential dimension of the sleeve opening may decrease, as indicated by the hashed lines 280.

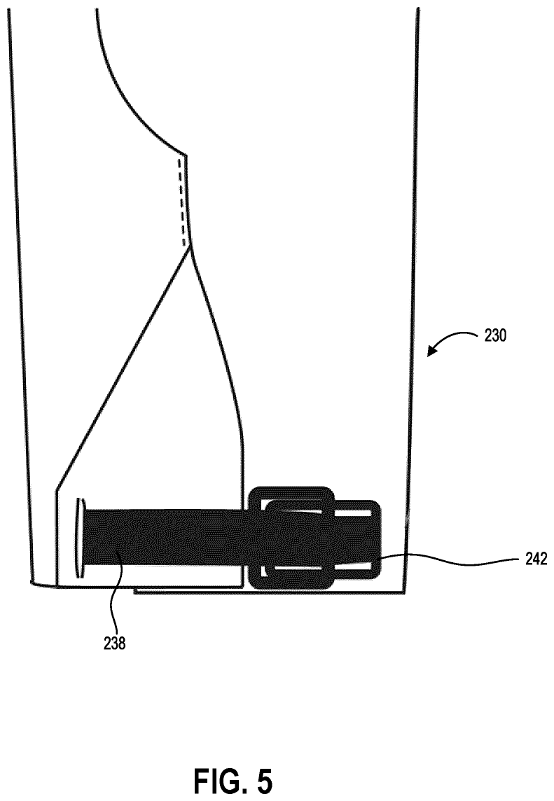

Reference is made to FIG. 5, which illustrates an enlarged, elevation view of the first sleeve portion 230 of FIG. 2. In FIG. 5, the fastener includes the elastomeric band 238 and the latch device 242. The elastomeric band 238 may be configured to mate with the latch device 242. The latch device 242 may be configured to transition the first sleeve portion 230 from an open state to a closed state, or vice versa, in a consistent way. When the first sleeve portion 230 is configured in the closed state, the sensor panel 240 may be positioned against the user limb with substantially consistent pressure.

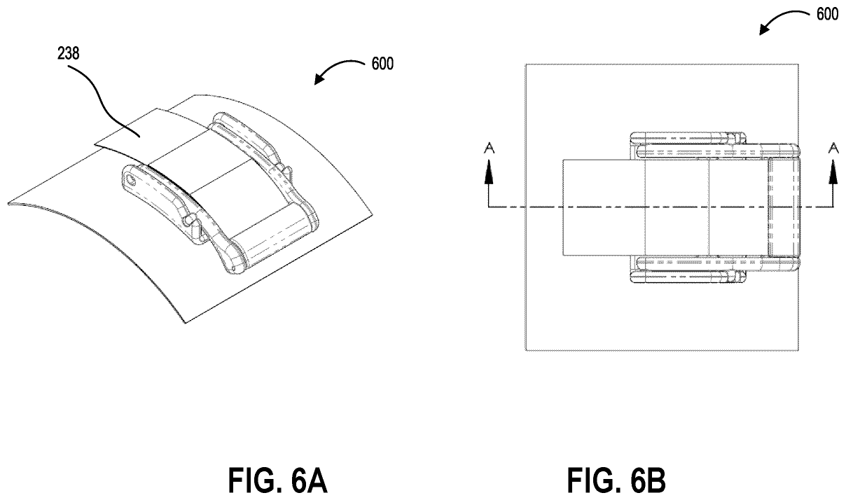

Reference is made to FIGS. 6A and 6B, which illustrate a perspective view and a top plan view of a latch 600 for a garment, respectively, in accordance with an embodiment of the present disclosure. As an example, the latch 600 may be the latch device 242 illustrated in FIG. 5.

In some embodiments, the latch 600 may be positioned on the bridge region 236 of the first sleeve portion 230 illustrated in FIG. 2. The latch 600 may be configured to receive or coupled to the elastomeric band 238 and may transition the first sleeve portion 230 from an open state to a closed state, or vice versa, in a consistent way.

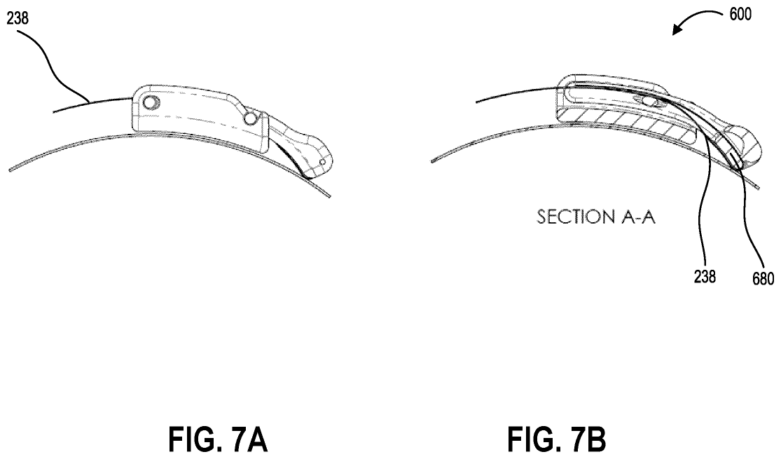

FIG. 7A illustrates a side elevation view of the latch 600 of FIGS. 6A and 6B. In FIG. 7A, the latch is in a closed state.

FIG. 7B illustrates a cross-sectional view of the latch 600 taken at line A-A illustrated in FIG. 6B of the latch 600. In FIG. 7B, the latch 600 is configured in the closed state and may tension or wrap the elastomeric band 238 in a configuration such that the circumferential dimension of the sleeve opening positions the sensor panel 240 (not illustrated in FIG. 7B) against the user limb with substantially consistent pressure. In FIG. 7B, a portion of the elastomeric band 238 is wrapped around or tucked within the latch lever 680.

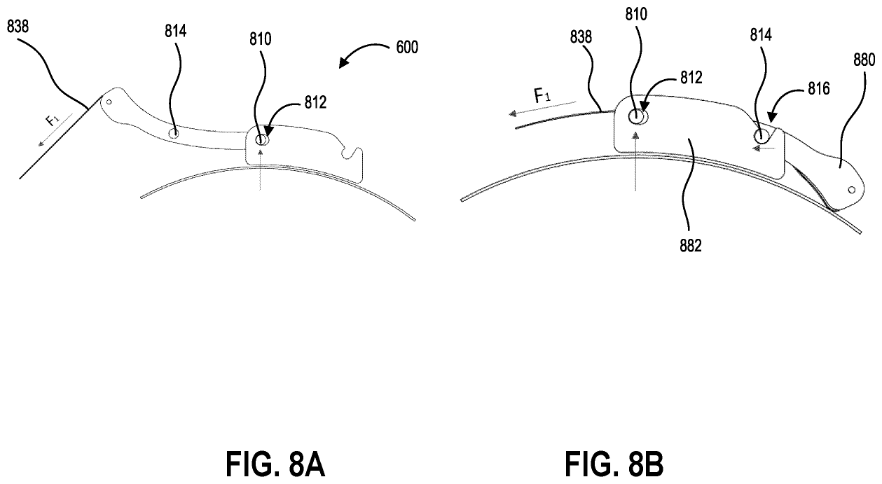

Reference is made to FIGS. 8A and 8B, which illustrate side elevation views of the latch 600 of FIG. 6A, 6B, 7A, or 7B, in accordance with embodiments of the present disclosure. FIG. 8A illustrates the latch 600 in an open state. When the latch 600 is in the open state, the latch pivot point 810 is biased to a first side of a pivot slot 812. The elastomeric band 838 may provide a force in a direction F1 to place the latch 600 in the open state and to bias the pivot point 710 to the first side of the pivot slot 710.

FIG. 8B illustrates the latch 600 in the closed state. When the latch 600 is in the closed state, a lever pin 814 may be received within a latch groove 816. The latch 600 may tension and wrap the elastomeric band 838 in a configuration such that the circumferential dimension of the sleeve opening may position the sensor panel 240 against the user limb with substantially consistent pressure.

When the latch 600 is in the closed state, the elastomeric band 838 may provide a force in the direction F1 such that the pivot point 810 may be biased to the first side of the pivot slot 812 and such that the lever pin 814 may be biased against a first side of the latch groove 816. Until the latch lever 880 is lifted away from the latch base 882, the elastomeric band 838 provides the force in the direction F1 to retain the latch in the closed state.

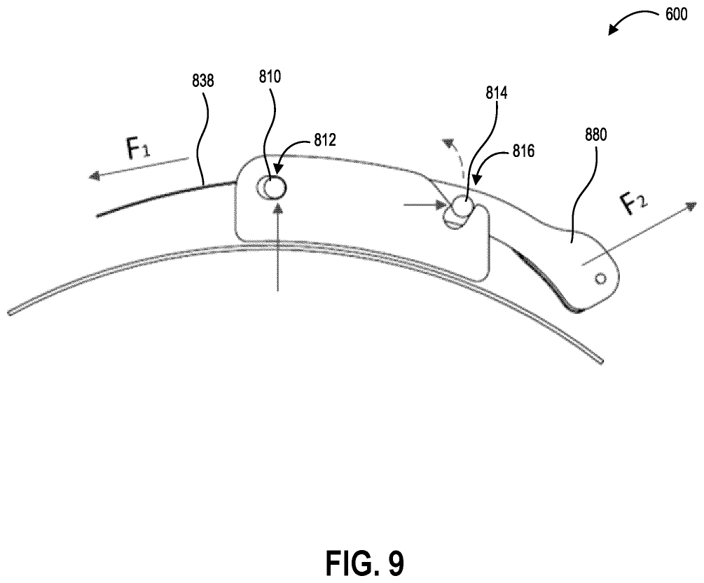

Reference is made to FIG. 9, which illustrates the latch 600 being transitioned from the closed state to the open state. When the user desires to transition the latch 600 to the open state the user may exert a force on the latch lever 880 in the direction indicated by the arrow F2. When the latch lever 880 is pushed in the direction indicated by the arrow F2, the latch pivot point 810 may be biased towards a second side of the pivot slot 812. Further, the lever pin 814 may be biased towards a second side of the latch groove 816, thereby causing the latch lever 780 to be lifted away from the latch base 782.

In the illustrated example states of the latch 600, the elastomeric band 838 may be consistently transitioned between two tensioned states for either: (1) positioning the sensor panel against the user limb (e.g., latch in closed state); or (2) releasing the sensor panel from the tensioned state from the user limb, such that the user limb may be re-positioned or removed from the sleeve provided by the first sleeve portion 230 (FIG. 2).

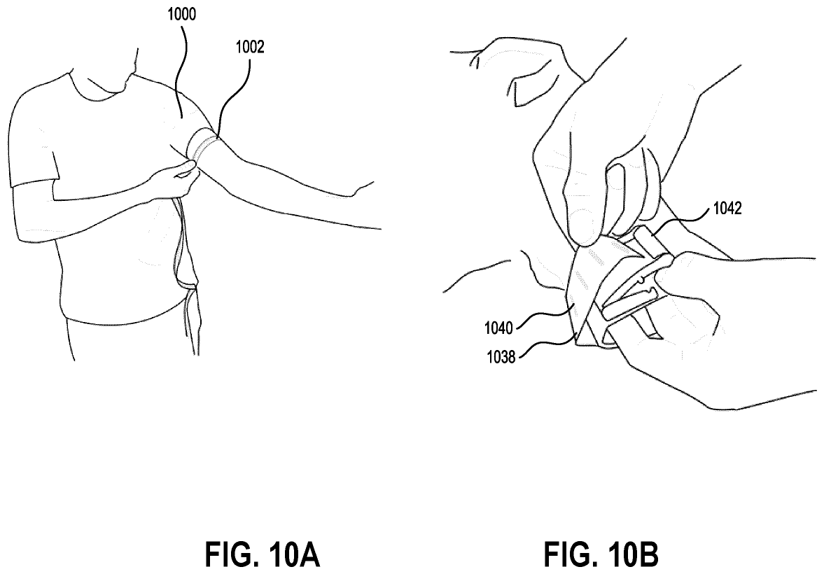

Reference is made to FIGS. 10A and 10B, which illustrate perspective views of a user 1000 conducting operations for calibrating a garment for detecting physiological data, in accordance with embodiments of the present disclosure.

In FIG. 10A, the user 1000 may obtain a measurement associated with a length around the user's upper arm. The user 1000 may obtain the measurement using a tape measure 1002 or other device for identifying a length measurement around the user's upper arm. The length measurement may correspond to the location at which the sensor panel may be positioned against when the user 1000 wears the garment having features described in the present disclosure.

The user 1000 may determine a gradation 1040 or size marking on an elastomeric band 1038 corresponding to the length measurement around the user's upper arm. The suitable gradation 1040 or size marking may be based on a determined look-up table associated with the garment having features described in the present disclosure.

The user 1000 may couple the elastomeric band 1038 to a latch 1042 at the previously determined suitable gradation 1040 or size marking on the elastomeric band 1038 for calibrating the garment for detecting physiological data for the user.

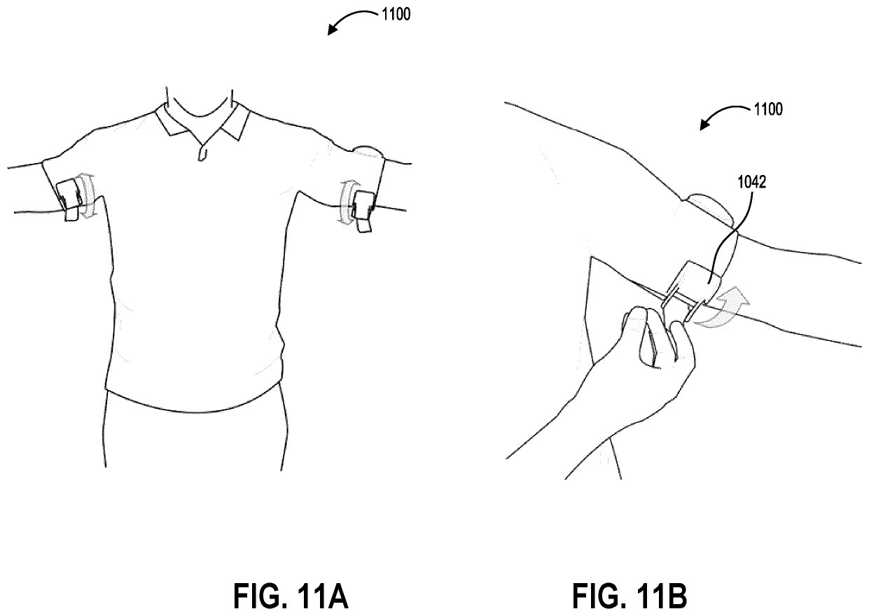

Reference is made to FIG. 11A, which illustrates a user 1100 wearing a garment for detecting physiological data, in accordance with an embodiment of the present disclosure. The garment illustrated in FIG. 11A includes one or more cuffs coupled to the garment sleeve opening having features described in the present disclosure. The cuff may be provided on each of the garment sleeve openings (e.g., on a left garment sleeve opening and on a right garment sleeve opening).

FIG. 11B illustrates a partial view of the user 1100 transitioning the latch 1042 from an open state to a closed state, such that a sensor panel may be positioned against the user limb with substantially consistent pressure.

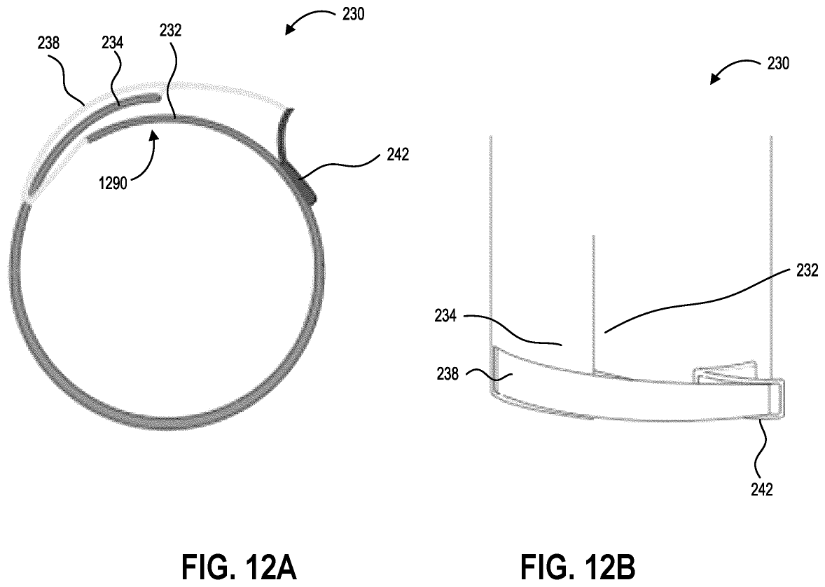

Reference is made to FIG. 12A, which illustrates a cross-sectional end view of an assembled first sleeve portion 230 of FIG. 2, in accordance with an embodiment of the present disclosure. The first sleeve portion 230 may be configured in an open state such that a user may readily insert a user limb into the shirt sleeve. The assembled first sleeve portion 230 may be a cuff coupled to the shirt sleeve opening. The cuff may include the elongate band having the first region 230 and the second region 234 and defining an adjustable loop for receiving the user limb based on the first region 230 being positioned proximal the second region 234.

In some embodiments, a stiffener member may be coupled to the first region 232 to define a substantially rigid base, and a sensor panel may be affixed to the substantially rigid base on a user limb facing side 1290 of the first region 232.

A fastener may include the elastomeric band 238 and the latch 242. The latch 242 may be coupled to the elastomeric band 238 and may transition the elongate band between a first adjustable loop circumference and a second adjustable loop circumference. In FIG. 12A, the latch 242 is illustrated in an open position, such that the user may readily insert or remove the user limb into or from the shirt sleeve.

The fastener may be coupled to the first region 232 and the second region 234 and may be configured to position a sensor panel (not illustrated in FIG. 12A) against the user limb with substantially consistent pressure when the latch 242 is placed in a closed position.

FIG. 12B illustrates a partial, perspective view of an assembled first sleeve portion 230, in accordance with an embodiment of the present disclosure. In FIG. 12B, the latch 242 may be in an open state or a partially open position.

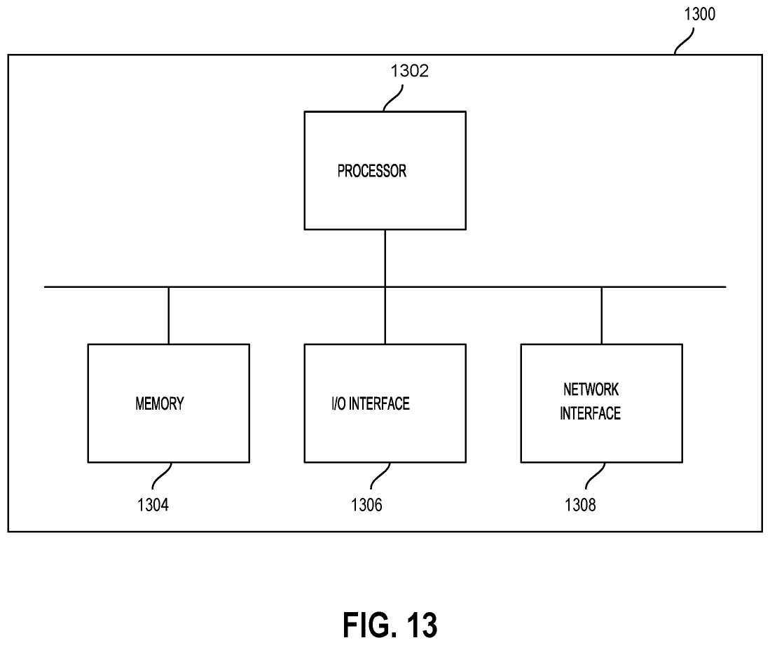

Reference is made to FIG. 13, which illustrates a block diagram of a computing device 1300, in accordance with an embodiment of the present disclosure. As an example, the controller device 100 of FIG. 1 may be implemented using the example computing device 1300 of FIG. 13.

The computing device 1300 includes at least one processor 1302, memory 1304, at least one I/O interface 1306, and at least one network communication interface 1308.

The processor 1302 may be a microprocessor or microcontroller, a digital signal processing (DSP) processor, an integrated circuit, a field programmable gate array (FPGA), a reconfigurable processor, a programmable read-only memory (PROM), or combinations thereof.

The memory 1304 may include a computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM).

The I/O interface 1306 may enable the computing device 1300 to interconnect with one or more input devices, such as a keyboard, mouse, camera, touch screen and a microphone, or with one or more output devices such as a display screen and a speaker.

The networking interface 1308 may be configured to receive and transmit data sets representative of the machine learning models, for example, to a target data storage or data structures. The target data storage or data structure may, in some embodiments, reside on a computing device or system such as a mobile device.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

1. A garment cuff for detecting physiological data comprising:
an elongate band having a first region and a second region and defining an adjustable loop for receiving a user limb based on the first region being positioned proximal the second region;
a stiffener member coupled to the first region to define a substantially rigid base;
a sensor panel affixed to the substantially rigid base on a user limb facing side of the first region; and
a fastener coupled to the elongate band and configured to position the sensor panel against the user limb with substantially consistent pressure.

2. The garment of claim 1, wherein the stiffener member is knitted to the first region of the elongate band.

3. The garment of claim 1, wherein the fastener includes:
an elastomeric band wrapping around the elongate band; and
a latch coupled to the elastomeric band, wherein the latch transitions the elongate band between a first adjustable loop circumference and a second adjustable loop circumference.

4. The garment of claim 1, wherein the sensor panel comprises a combination of data acquisition sensors including an electrocardiogram (ECG) sensor and a photoplethysmogram (PPG) sensor.

5. The garment of claim 1, wherein the sensor panel comprises a ballistocardiography (BCG) sensor.

6. The garment of claim 1, comprising: a two-way stretch fabric coupled to the second region.

7. The garment of claim 1, comprising a 1-way stretch fabric joining the first region and the second region, wherein the 1-way stretch fabric stretches in a direction between the first region and the second region.

8. The garment of claim 1, comprising: a data acquisition module coupled to the sensor panel for receiving physiological data from the sensor panel.

9. The garment of claim 8, wherein the data acquisition module is removably mounted on a bridge region between the first region and the second region of the elongate band.

10. The garment of claim 1, wherein the garment includes a shirt, and wherein the cuff is coupled to a shirt sleeve of the shirt.

11. The garment of claim 1, wherein the garment includes at least one of pants or shorts, and wherein the cuff is coupled to a pant leg.

12. A garment for detecting physiological data comprising:
a garment body; and
a cuff coupled to the garment body providing a garment opening for receiving a user limb, the cuff comprising:
an elongate band having a first region and a second region and defining an adjustable loop for receiving a user limb based on the first region being positioned proximal the second region;
a stiffener member coupled to the first region to define a substantially rigid base;
a sensor panel affixed to the substantially rigid base on a user limb facing side of the first region; and
a fastener coupled to the elongate band and configured to position the sensor panel against the user limb with substantially consistent pressure.

13. The garment of claim 12, wherein the stiffener member is knitted to the first region of the elongate band.

14. The garment of claim 12, wherein the fastener includes:
an elastomeric band wrapping around the elongate band; and
a latch coupled to the elastomeric band, wherein the latch transitions the elongate band between a first adjustable loop circumference and a second adjustable loop circumference.

15. The garment of claim 12, wherein the sensor panel comprises a combination of data acquisition sensors including an electrocardiogram (ECG) sensor and a photoplethysmogram (PPG) sensor.

16. The garment of claim 12, wherein the sensor panel comprises a ballistocardiography (BCG) sensor.

17. The garment of claim 12, comprising: a two-way stretch fabric coupled to the second region.

18. The garment of claim 12, comprising a 1-way stretch fabric joining the first region and the second region, wherein the 1-way stretch fabric stretches in a direction between the first region and the second region.

19. The garment of claim 12, comprising: a data acquisition module coupled to the sensor panel for receiving physiological data from the sensor panel.

20. The garment of claim 19, wherein the data acquisition module is removably mounted on a bridge region between the first region and the second region of the elongate band.
